(19) United States
(12) Patent Application Publication
(10) Pub. No.: US 2013/0000638 A1
(43) Pub. Date: Jan. 3, 2013

(54) ALUMINUM CYLINDER FOR NO/NITROGEN GAS MIXTURE AND USE THEREOF IN THE TREATMENT OF PULMONARY VASOCONSTRICTIONS

(75) Inventors: Pierre DE VILLEMEUR, Louveciennes (FR); Laurent LECOURT, Boulogne (FR); Bénédicte LEVINSON, Levallois Perret (FR)

(73) Assignees: Air Liguide Sante (International), Paris (FR); L'Air Liquide, Societe Anonyme pour I'Etude et I'Exploitation des Procedes Georges claude, Paris (FR)

(21) Appl. No.: 13/537,808

(22) Filed: Jun. 29, 2012

(30) Foreign Application Priority Data
Jun. 29, 2011 (FR) 1155789

(51) Int. Cl. F17C 1/14; A61M 16/12; F17C 5/06

(52) U.S. Cl. 128/203.12; 220/581; 141/4

(57) ABSTRACT

The invention relates to a container for packaging NO/N2 mixtures, such as a gas cylinder, comprising a main body (1) comprising an internal volume (7) having a capacity between 2 and 30 liters and containing an NO/N2 mixture at a pressure above atmospheric pressure, the main body (1) being formed from an aluminum alloy, characterized in that the main body (1) is formed from an aluminum alloy comprising aluminum and a proportion by weight of Cr of between 0.05 and 0.80%, of Cu of between 1 and 3%, of Pb of at most 100 ppm, of Zn of between 5 and 8%, and of Mg of between 1 and 3%.

### CROSS REFERENCE TO RELATED APPLICATIONS

This application claims the benefit of priority under 35 U.S.C. §119 (a) and (b) to French Application No. 1155789, filed Jun. 29, 2011, the entire contents of which are incorporated herein by reference.

### BACKGROUND

The invention relates to a container for packaging NO/N₂ mixtures comprising a main body formed from aluminum or from an aluminum alloy, in particular a gas cylinder of cylindrical shape, and the use thereof in the treatment of pulmonary vasoconstrictions in adults or children.

NO/N₂ gas mixtures are commonly used for treating pulmonary vasoconstrictions in adults or children, in particular in newborns suffering from primary pulmonary hypertension or in patients undergoing a cardiac surgery operation.

These NO/N₂ mixtures are conventionally packaged into steel gas cylinders. Typically these cylinders contain from 100 to 1000 ppm by volume of NO, the remainder being nitrogen (N₂). These cylinders customarily have a water capacity of 2 to 50 liters, which makes it possible to introduce therein a total charge that may range up to 15 m³ of NO/N₂ mixture.

The packaging of these mixtures, i.e. the filling of cylinders with these mixtures, is carried out at gas filling centers.

However, in practice an inaccuracy has been observed during the packaging of NO/N₂ gas mixtures in these steel cylinders. Specifically, a difference has been noted between the internal volume of each cylinder available for the packaging of NO compared to the theoretical water volume of these cylinders; this difference is referred to hereinbelow as “capacity difference”.

This capacity difference has no influence on the final NO content but has repercussions on the final charge of the cylinder, which is then either greater than or less than the desired charge, i.e. compared to the set point charge.

However, this capacity difference is not negligible since it may reach around 5% of the total gas charge of each cylinder.

Furthermore, the packaging containers must also not be of excessive weight, and must above all withstand impacts and a gas pressure that may reach several hundreds of bar, typically from 200 to 300 bar, and also withstand corrosion.

### SUMMARY

The objective of the present invention is to solve these problems, i.e. to minimize said capacity difference by obtaining a final charge as close as possible to the charge set point, i.e. a difference of the order of ±1.5% or lower, while proposing a packaging container that is resistant to impacts and to high pressures, and also to corrosion, but without having too high a weight.

The solution of the invention is a container for packaging NO/N₂ mixtures comprising a main body comprising an internal volume having a capacity between 2 and 30 liters and containing an NO/N₂ mixture at a pressure above atmospheric pressure, the main body being formed from an aluminum alloy, characterized in that the main body is formed from an aluminum alloy comprising predominantly, i.e. more than 50% by weight, or even more than 60% by weight, preferably more than 75% by weight, aluminum and a proportion by weight of Cr of between 0.05 and 0.80%, of Cu of between 1 and 3%, of Pb of at most 100 ppm, of Zn of between 5 and 8%, and of Mg of between 1 and 3%.

Within the context of the present invention, it has been demonstrated that the difference in charge, i.e. the inaccuracy of the charge volume, was in fact linked to the cylinders used for packaging the NO/N₂ mixtures. Specifically, it has been observed that the use of steel cylinders did not make it possible to obtain a desired charge accuracy but that, conversely, the use of aluminum cylinders did make it possible to obtain the desired accuracy.

However, although the fact of producing the cylinders in aluminum also makes it possible to provide a relative lightness, it was observed that the aluminum alloy must be chosen carefully so as not to generate excessive brittleness of the cylinders. This brittleness may be avoided or minimized by controlling especially the chosen aluminum alloy composition, i.e. the content of the various elements of the alloy, in particular that of Mg, Cr, Cu and/or Zn, but also the lead content of the alloy and secondarily the grain size of the alloy.

Depending on the case, the packaging container of the invention may comprise one or more of the following technical features:
The aluminum alloy comprises (% by weight) less than 0.5% of Mn, less than 0.3% of Fe, less than 0.2% of Si and/or less than 0.2% of Ti.
The aluminum alloy comprises (% by weight) at most 50 ppm of Pb, preferably at most 30 ppm of Pb.
The aluminum alloy comprises (% by weight) between 0.1 and 0.50% of Cr, between 1.5 and 2.8% of Cu, between 5.4 and 7.6% of Zn and/or between 1.2 and 2.8% of Mg.
The aluminum content of the aluminum alloy is between 85 and 93% by weight.
The aluminum alloy comprises (% by weight) between 0.12 and 0.30% of Cr, between 1.6 and 2.7% of Cu, and/or between 1.25 and 2.6% of Mg.
The aluminum alloy comprises (% by weight) between 0.15 and 0.25% of Cr, between 1.7 and 2.6% of Cu, and/or between 1.3 and 2.5% of Mg.
The aluminum alloy comprises (% by weight) between 5.5 and 7.5% of Zn.
The aluminum alloy comprises (% by weight) less than 0.3% of Mn, preferably less than 0.2% of Mn.
The aluminum alloy comprises (% by weight) less than 0.2% of Fe.
The aluminum alloy comprises (% by weight) less than 0.15% of Si.
The aluminum alloy comprises (% by weight) less than 0.15% of Ti, preferably less than 0.10% of Ti.
The aluminum alloy comprises (% by weight) less than 0.02% of Bi, preferably less than 0.01% of Bi.
The aluminum alloy comprises (% by weight) less than 0.01% of Zr, preferably less than 0.05% of Zr.
The maximum grain size of the alloy should not normally exceed 1 mm, or even 0.56 mm in certain cases (2-liter cylinders for example). The grain size requirement applies to all the grains and not to the mean grain size.
It is a gas cylinder comprising a main body of cylindrical shape.
It has a capacity (water volume) between 2 and 25 liters.
It has a diameter between 10 and 30 cm, and a height between 50 and 120 cm approximately.
The main body comprises a neck with an outlet orifice, attached to which is a device for controlling the passage of gas and/or for reducing the pressure.
The body comprises a peripheral wall having a thickness between 2 and 20 mm.
The NO/N₂ mixture contains from 1 to 1000 ppm by volume of NO, the remainder being nitrogen (N₂).

The invention also relates to a process for packaging an NO/N₂ mixture in a packaging container according to the invention, comprising a main body comprising an internal volume, introduced into which is the NO/N₂ mixture at a pressure above atmospheric pressure.

Furthermore, the invention also relates to the use of a container according to the invention comprising a main body comprising an internal volume, said main body being formed from aluminum or from an aluminum alloy, for packaging an NO/N₂ gas mixture containing from 1 to 1000 ppm by volume of NO, the remainder being nitrogen.

Moreover, the invention also relates to a system for packaging an NO/N₂ gas mixture comprising a container according to the invention.

Furthermore, the invention also relates to an installation for dispensing NO/N₂ mixtures that can be used in the context of treating of pulmonary hypertension in adults, children or newborns, comprising a ventilator that delivers an oxygen-containing gas, such as air or an oxygen/nitrogen mixture, having a patient circuit connecting said ventilator to a patient interface, for example a respiratory mask or a cannula, characterized in that said patient circuit is supplied with gas by a container according to the invention, in particular a gas cylinder. Preferably, the gas passes through a control device, such as the Opti Kinox device from Air Liquide Santé, placed between the container(s) and the patient circuit.

Finally, the invention also relates to an NO/N₂ gas mixture for use for treating or for preventing pulmonary vasoconstriction or pulmonary hypertension in adults, children or newborns, in which the NO/N₂ mixture is administered by inhalation, advantageously using an installation for dispensing an NO/N₂ mixture comprising a ventilator having a patient circuit connecting said ventilator to a patient interface, said patient circuit being supplied with an NO/N₂ gas mixture delivered by a container according to the invention, in particular a gas cylinder, the main body of which is formed from an aluminum alloy comprising aluminum (predominantly) and a proportion by weight of Cr of between 0.05 and 0.80%, of Cu of between 1 and 3%, of Pb of at most 100 ppm, of Zn of between 5 and 8%, and of Mg of between 1 and 3%.

According to the invention, the NO/N₂ gas mixture is used for treating a newborn suffering from primary pulmonary hypertension or an adult, a child or a newborn who must undergo, is undergoing or has undergone a cardiac surgery operation.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 schematically shows a gas container according to the invention (seen in cross section); and

FIG. 2 schematically shows an installation for dispensing NO/N₂ mixtures comprising a cylinder of NO/N₂ according to FIG. 1.

### DESCRIPTION OF PREFERRED EMBODIMENTS

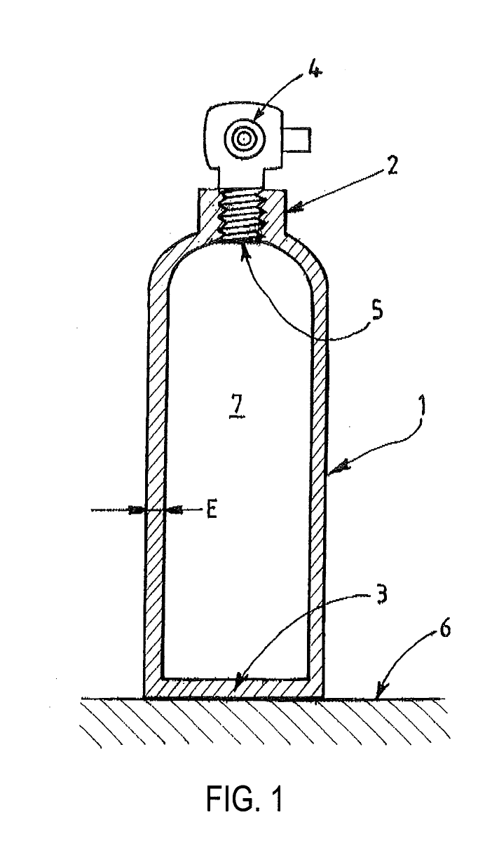

The appended FIG. 1 shows one embodiment of a container according to the invention (seen in cross section), namely a cylinder for packaging NO/nitrogen gas mixtures which is composed of a main body 1 of general cylindrical shape comprising a narrow section or neck 2 at which there is the opening 5 for passage of the gas and, at its other end, a flat base 3 on which the cylinder rests when it is placed on a support, such as the floor 6.

This cylinder comprises an internal volume 7 having a typical capacity:
either of 5 liters (of water), a diameter of 14 cm, and a height of 63 cm,
or of 20 liters (of water), a diameter of 21 cm, and a height of 108 cm.

It is however also possible to have a cylinder of smaller capacity, for example of 2 liters only, or of different dimensions.

An NO/nitrogen mixture containing up to 1000 ppm by volume of NO and nitrogen for the remainder, for example 225, 450 or 800 ppm vol., is packaged in the cylinder at a pressure of more than 150 bar, for example 180 bar.

A valve 4 or an integrated valve regulator is mounted, for example screwed, into the neck 2 of the cylinder so as to control the flow rate and/or the pressure of the gas leaving the container.

In accordance with the invention, the body 1 of the cylinder is formed from an aluminum alloy, for which examples of compositions of alloys A and B are given in the table below.

TABLE

| | Proportion by weight | Proportion by weight |
|---|---|---|
| Elements | Alloy A | Alloy B |
| Pb | <30 ppm | <100 ppm |
| Fe | <0.2% | <0.12% |
| Si | <0.15% | <0.1% |
| Cu | 1.8-2.6% | 1.7-2.3% |
| Mn | <0.2% | <0.05% |
| Mg | 1.3 to 2.1% | 1.5-2.5% |
| Cr | 0.15 to 0.25% | 0.15 to 0.25% |
| Zn | 6.1-7.5% | 5.5-6.5% |
| Ti | <0.05% | <0.1% |
| Zr | <0.05% | / |
| Bi | / | <0.01% |
| Impurities | 0.15% max | |
| Al | 86.7-90.7% | 87.9-91.2% |

By using a cylinder with a body 1 made of aluminum alloy, as given in particular in the preceding table, it is possible to minimize the aforementioned capacity difference by obtaining a final charge as close as possible to the charge set point, i.e. a difference of the order of ±1.5% or lower, while having a container that is resistant to impacts, to high pressures and to corrosion.

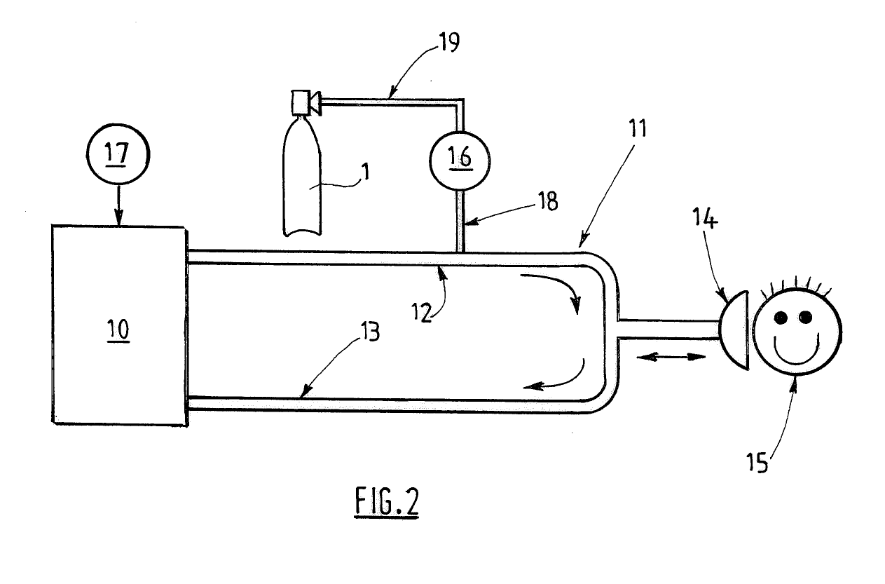

The appended FIG. 2 illustrates one embodiment of an installation for dispensing NO/N₂ mixtures comprising a ventilator 10 having a patient circuit 11 connecting the ventilator 10 to a patient interface 14. The patient circuit 11 is supplied with gas by a container 1 according to FIG. 1, the composition of which is for example that of the alloy A or B from the preceding table.

The ventilator 10 therefore supplies with NO-enriched respiratory gas, a respiratory circuit or patient circuit 11 with two branches, i.e. with an inhalation branch 12 and an exhalation branch 13. The inhalation branch 12 is designed to convey a respiratory gas, for example air, oxygen-enriched air, oxygen or a nitrogen/O₂ mixture, from the ventilator 10 to the patient 15, whilst the exhalation branch 13 is designed to convey the gas exhaled by the patient 15, therefore rich in CO₂, to the ventilator 10.

At the patient 15, the gas is administered by means of a patient interface 14, for example a respiratory mask, a catheter or a tracheal cannula or any other suitable gas administration device.

The ventilator 1 is supplied with an oxygen-containing gas, for example air (O₂ content of 21% by volume), oxygen-enriched air (O₂ content >21% by volume) or pure oxygen (100% O₂).

The oxygen-containing gas originates from a gas source 17, such as one or more gas cylinders, a gas mixer within which the mixing, for example, of air and oxygen, is carried out, or one or more gas lines transporting one or more gases, for example a line transporting oxygen from an oxygen production unit, such as a pressure swing (PSA) unit, or an oxygen storage unit, such as a buffer or storage tank.

As already stated, the oxygen-rich gas is delivered by the ventilator 10 into the inhalation branch 12 of the patient circuit 11.

Furthermore, a device 16 for dispensing NO is fluidically connected to said inhalation branch 12 of the patient circuit 11 in order to deliver thereto, via a supply line 18, an NO/N₂ mixture, for example formed of 400, 800 or 1000 ppmv of NO, the remainder being nitrogen.

The device 16 for dispensing NO is itself supplied with NO/N₂ mixture, via a gas feed line 19, by an NO/N₂ container 1 that is part of a storage and dispensing system according to the invention.

The NO/N₂ container 1 is a gas cylinder made of aluminum alloy (alloy A or B for example) with a capacity of 2, 5 or 11 liters (water equivalent), equipped with an integrated valve regulator, preferably protected by a cap for protection against impacts. The integrated valve regulator, also referred to as “IVR”, makes it possible to control the outlet of gas from the container 1 and its outlet pressure. Control members make it possible to control the release of the gas and furthermore to regulate the outlet pressure level.

Such an installation is suitable for the treatment, with administration of NO by inhalation, of pulmonary vasoconstrictions or pulmonary hypertension in a patient 15, who may be an adult, a child or a newborn.

More specifically, the NO/N₂ gas mixture transported to the patient 15 by the patient circuit 11 and dispensed by means of the interface 14, is particularly recommended for treating a newborn suffering from primary pulmonary hypertension or an adult, a child or a newborn who must undergo, is undergoing or has undergone a cardiac surgery operation.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A container for packaging NO/N₂ mixtures comprising a main body (1) comprising an internal volume (7) having a capacity between 2 and 30 liters and containing an NO/N₂ mixture at a pressure above atmospheric pressure, the main body (1) being formed from an aluminum alloy, wherein the main body (1) is formed from an aluminum alloy comprising predominantly aluminum and a proportion by weight of:
Cr of between 0.05 and 0.80%,
Cu of between 1 and 3%,
Pb of at most 100 ppm,
Zn of between 5 and 8%, and
Mg of between 1 and 3%.

2. The container of claim 1, wherein the aluminum content of the aluminum alloy is between 85 and 93% by weight.

3. The container of claim 1, wherein the container is a gas cylinder comprising a main body (1) of cylindrical shape and having a flat base and having a diameter between 10 and 30 cm, and a height between 50 and 120 cm.

4. The container of claim 1, wherein the main body (1) comprises a neck (2) with an outlet orifice (5), attached to which is a device (4) for controlling the passage of gas and/or for reducing the pressure.

5. The container of claim 1, wherein the NO/N₂ gas mixture contains from 1 to 1000 ppm by volume of NO, the remainder being nitrogen (N₂).

6. A process for packaging an NO/N₂ mixture in a packaging container of claim 1, comprising a main body (1) comprising an internal volume (7), introduced into which is the NO/N₂ mixture at a pressure above atmospheric pressure.

7. The installation for dispensing NO/N₂ mixtures comprising a ventilator having a patient circuit connecting said ventilator to a patient interface, wherein said patient circuit is supplied with gas by a container of claim 1.

8. A method for treating or for preventing pulmonary vasoconstriction or pulmonary hypertension in adults, children or newborns, comprising administering a NO/N₂ mixture by inhalation to a patient, said NO/N₂ gas mixture being delivered by a container of claim 1 within which the NO/N₂ mixture is packaged.

9. The method of claim 10, wherein the NO/N₂ mixture is administered by inhalation using an installation for dispensing an NO/N₂ mixture comprising a ventilator having a patient circuit connecting said ventilator to a patient interface, said patient circuit being supplied with an NO/N₂ gas mixture delivered by a container for packaging NO/N₂ mixtures comprising a main body (1) comprising an internal volume (7) having a capacity between 2 and 30 liters and containing an NO/N₂ mixture at a pressure above atmospheric pressure, the main body (1) being formed from an aluminum alloy, wherein the main body (1) is formed from an aluminum alloy comprising predominantly aluminum and a proportion by weight of:
Cr of between 0.05 and 0.80%,
Cu of between 1 and 3%,
Pb of at most 100 ppm,
Zn of between 5 and 8%, and
Mg of between 1 and 3%.

10. The method of claim 10, wherein the patient is a newborn suffering from persistent pulmonary hypertension (PPHN) or an adult, a child or a newborn who must undergo, is undergoing, or has undergone a cardiac surgery operation.
